(19) United States
(12) Patent Application Publication
(10) Pub. No.: US 2019/0175526 A1
(43) Pub. Date: Jun. 13, 2019

(54) METHODS FOR TREATING CANCER

(71) Applicant: Macau University of Science and Technology, Macau (CN)

(72) Inventors: Xiao Jun YAO, Macau (CN); Lai Han Elaine LEUNG, Macau (CN); Liang LIU, Macau (CN); Jia Hui XU, Macau (CN); Qian Qian WANG, Macau (CN); Ying LI, Macau (CN)

(21) Appl. No.: 15/839,883

(22) Filed: Dec. 13, 2017

(51) Int. Cl. A61K 31/136; A61P 35/00; G01N 33/574

(52) U.S. Cl. CPC A61K 31/136; A61P 35/00; G01N 2333/91017; G01N 33/57423; G01N 33/57496

(57) ABSTRACT

One embodiment relates to a method of treating cancer by administering a compound of Formula I to a patient. Another embodiment relates to a method of treating cancer with overexpressed protein arginine methyltransferases that includes administering to a patient the compound represented by the Formula I:

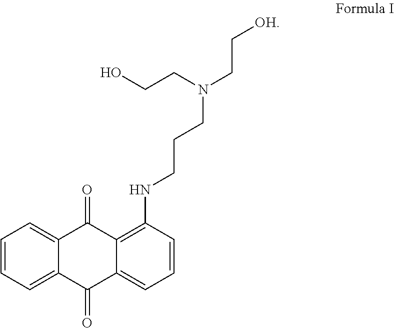 [SMILES: O=C1c2ccccc2C(=O)c2c(NCCCN(CCO)CCO)cccc21]

### FIELD OF THE INVENTION

The present invention relates to a method of treating cancer. In particular, the present invention relates to a method of treating cancer with overexpressed protein arginine methyltransferases.

### BACKGROUND OF INVENTION

Cancer is a disease involving abnormal cell growth with the potential to invade or spread to other areas of the body. Some cancers have overexpressed protein arginine methyltransferases (PRMTs). In particular, the overexpressed PRMT is protein arginine methyltransferase 5 (PRMT5).

In view of the demand for effectively treating cancer, particularly cancer with overexpressed PRMTs, improvements in method are desired.

### SUMMARY OF INVENTION

One example embodiment is a method of treating a cancer in a patient in need thereof. The method includes administering a therapeutically effective amount of a compound to the patient to treat the cancers, the compound is represented by Formula I:

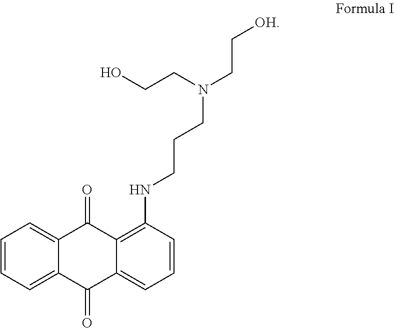 [SMILES: O=C1c2ccccc2C(=O)c2c(NCCCN(CCO)CCO)cccc21]

Another example embodiment is a method of treating cancer in a patient in need thereof. The method includes diagnosing the patient having a cancer with overexpressed protein arginine methyltransferases; and administering a therapeutically effective amount of a compound to the patient to treat the cancer, and the compound is represented by Formula I.

Other example embodiments are discussed herein.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1a shows a crystal structure of PRMT5-SAM-EPZ015666 (PDBID: 4×61) in accordance with an example embodiment.

FIG. 1b shows a binding mode between PRMT5 and Formula I (i.e. compound 3039-0164) in accordance with an example embodiment.

FIG. 1c shows a binding mode between PRMT5 and EPZ015666 which is an inhibitor of PRMT5 in accordance with an example embodiment.

FIG. 2a shows that compound 3039-0164 decreases cell viability of cell line A459 by MTT assay in accordance with an example embodiment.

FIG. 2b shows that compound 3039-0164 decreases cell viability of cell line H460 by MTT assay in accordance with an example embodiment.

FIG. 3 shows that compound 3039-0164 inhibits the methyltransferase activity of PRMT5 in accordance with an example embodiment.

FIG. 4 shows that compound 3039-0164 decreases the expression of H3R8me2s in cell line A549 after being treated with compound 3039-0164 for 24 hours in accordance with an example embodiment. Untreated cells were used as a control.

FIG. 5 shows that compound 3039-0164 decreases the protein expression of oncogene FGFR3 and eIF4E in cell line A549 after being treated with compound 3039-0164 for 24 hours in accordance with an example embodiment. Untreated cells were used as a control.

FIG. 6 shows that compound 3039-0164 decreases the levels of phosphorylated AKT, ERK and mTOR in cell line A549 after being treated with compound 3039-0164 for 24 hours in accordance with an example embodiment.

FIG. 7 shows a method to treat cancer in a patient in accordance with an example embodiment.

FIG. 8 shows a method to treat cancer in a patient in need of such treatment in accordance with an example embodiment.

FIG. 9 shows a method to inhibit progress of tumor growth in a patient with cancer in accordance with an example embodiment.

### DETAILED DESCRIPTION OF THE PREFERRED EMBODIMENTS

Example embodiments relate to methods of treating a cancer in a patient in need thereof. The methods comprise administering a therapeutically effective amount of a compound represented by Formula I (i.e. compound 3039-0164):

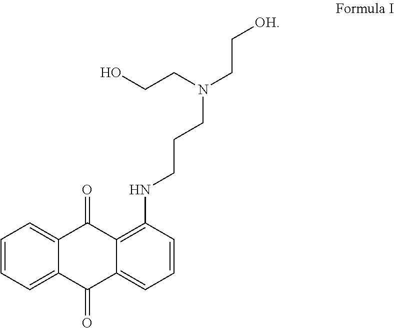 [SMILES: O=C1c2ccccc2C(=O)c2c(NCCCN(CCO)CCO)cccc21]

Example embodiments relate to methods of treating cancer in a patient in need thereof. The methods comprise diagnosing the patient having a cancer with overexpressed protein arginine methyltransferases; and administering a therapeutically effective amount of a compound represented by Formula I to the patient to treat the cancer.

Example embodiments relate to a pharmaceutical composition comprising a compound and a pharmaceutically acceptable excipient. The compound is represented by I.

In one example embodiment, the cancer is lymphomas, breast cancer, lung cancer, colorectal cancer or glioblastoma. In a further embodiment for example, the lung cancer is non-small cell lung cancer (NSCLC). In yet another example embodiment, the lung cancer is lung adenocarcinoma.

In one example embodiment, the cancer has overexpressed protein arginine methyltransferases (PRMTs). In a further embodiment for example, the protein arginine methyltransferase is protein arginine methyltransferase 5 (PRMT5). In yet another example embodiment, Formula I inhibits PRMT5.

PRMTs are a class of enzymes that transfer a methyl group from the cofactor S-adenosylmethionine (SAM) onto arginine omega nitrogen of substrate protein especially for histone. Based on product specificity, PRMTs can be divided into three subclasses: type I, II and III, which asymmetrically dimethylate, symmetrically dimethylate, and monomethylate their substrates, respectively. PRMT5 as a type II PRMT, catalyzes the symmetrical dimethylation of arginine residues of histone, and plays multiple roles in cellular processes including differentiation, proliferation, apoptosis and ribosome biogenesis. For instance, PRMT5-driven methylation of arginine residues leads to symmetric dimethylation of histone H3 (H3R8me2s), which in turn alters chromatin structure to promote transcriptional repression.

### Example 1

### Material and Methods

### 1. Cell Culture and Cell Viability Assays

Cell lines A549 and H460 were purchased from ATCC and cultivated in RPMI 1640 medium supplemented with 10% FBS (Gibco, Big Cabin, Okla., Me., USA), 1% penicillin-streptomycin solution and maintained at 37° C. in a CO₂ incubator with 5% CO₂. Compound 3039-0164 was dissolved in DMSO and stored at −40° C. Cells were seeded on a 96-well microplate with 3,000 cells/well, cultured overnight for cell adhesion, and treated with DMSO or various concentrations of compound 3039-0164 for 72 hours. Then, each well was added with 10 μL MTT (5 mg/mL) and incubated for 4 hours at 3° C., followed by adding 100 μL acidic isopropanol (10% SDS and 0.01 mol/L HCl). Finally, an absorbance at 570 nm was measured by a microplate reader (Tecan, Morrisville, N.C., USA). Cell viability was calculated relative to untreated controls, with results based on at least three independent experiments.

### 2. In Vitro Enzymatic Assays

PRMT5 enzymatic assay was carried out by Shanghai ChemPartner Co. (998 Halei Road, Pudong New Area, Shanghai, 201203, China). To measure IC₅₀ values, 10 concentrations of compound 3039-0164 were tested. PRMT5 was purchased from BPS bioscience (Cat. No. 51045), and SAM/SAH were purchased from Sigma. Inc (Cat. No. A7007-100MG and No. A9384-25MG). Compound 3039-0164 was prepared as 10 mM stock in DMSO and diluted to final concentration in DMSO. PRMT5 and substrates were incubated with indicated concentrations of compounds in a 384-well plate for 60 minutes at room temperature. Then, acceptor and donor solutions were added to label the residual substrates of PRMT5. The labeling process lasted for 60 minutes at room temperature, followed by reading endpoint with EnSpire Plate Reader with Alpha mode (PerkinElmer).

### 3. Western Blot Analysis

Cells were washed twice with cold PBS, and lysed in RIPA lysis buffer containing protease and phosphatase inhibitors. Cell lysates were centrifuged for 5 minutes (at 12,000 g, 4° C.), and the supernatant was collected. Protein concentrations were determined by Bio-Rad Protein Assay Kit (Bio-Rad, Philadelphia, Pa., USA). Equal amounts of protein (50 μg) were separated on a 10% SDS-PAGE gel, and transferred to a nitrocellulose (NC) membrane at 300 mA and 4° C. for 1 h. The membrane was incubated with primary antibody (1:1000), and then with a fluorescence-conjugated secondary antibody (1:10000). GAPDH was used as the loading control and for normalization. The signal of membranes was scanned with LI-COR Odessy scanner (Befast, Me., USA).

### 4. Molecular Docking

Molecular docking calculation is performed to study the interaction between Formula I and PRMT5. A crystal structure of PRMT5 complexed with SAM and EPZ015666 (PDB ID:4×61) was used for modeling a binding mode between Formula I and PRMT5. After the preparation for this complex with Prep Wiz in Maestro (version 10.2, Schrodinger), a grid file was generated based on the position of EPZ015666 in Grid Generation wizard for docking. Formula I was then preprocessed by LigPrep under OPLS-2005 force field, and the low-energy three dimensional conformers were created. Finally, Glide with the standard precision (SP) scoring mode was used to dock Formula I into PRMT5 binding pocket. The pose with the lowest docking score was chosen for further binding mode analysis.

### 5. Statistical Analysis

Descriptive analytical data were presented as means±SEM. Multiple comparisons were evaluated by one-way analysis of variance (ANOVA) using Graph Prim 5.0. P<0.05 was considered statistically significant.

### Example 2

### 1. Compound 3039-0164 Inhibits Cell Proliferation of NSCLC Cells

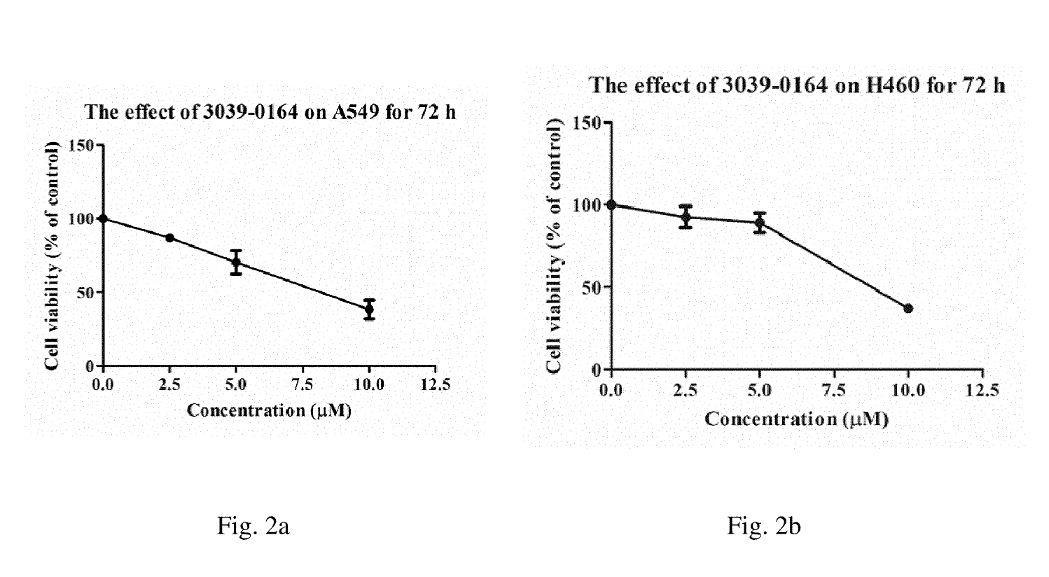

Two human non-small cell lung cancer (NSCLC) cell lines, A549 and H460, were treated with an increasing concentration (0, 2.5, 5.0, 7.5 and 10.0 μM) of compound 3039-0164 for 72 hours, and then cell viability was determined by MTT assay. As shown in FIG. 2a and FIG. 2b, the cell viability of A549 and H460 were both decreased by compound 3039-0164 in a dose-dependent manner. Table 1 shows that the inhibition concentration (IC₅₀) values of compound 3039-0164 on cell lines A459 and H460 are 7.79±1.80 and 8.64±1.46 μM, respectively. It suggested that compound 3039-0164 exhibited significantly cytotoxic effect on NSCLC A549 and H460 cells.

TABLE 1
IC₅₀ values of compound 3039-0164 one cell lines A459 and H460

| Cell Lines | IC₅₀ (μM) |
|---|---|
| A459 | 7.79 ± 1.80 |
| H460 | 8.64 ± 1.46 |

### 2. Compound 3039-0164 Inhibits the Methyltransferase Activity of PRMT5

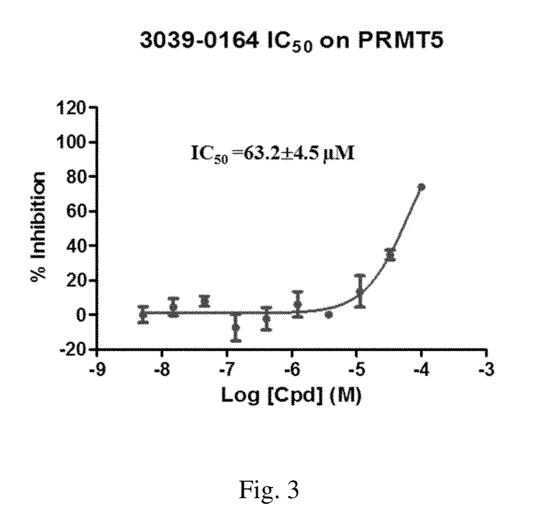

To investigate the influence of compound 3039-0164 on enzymatic activity and its “on-target” effect, AlphaLISA assay was carried out. The result showed that compound 3039-0164 inhibited PRMT5 enzyme activity in a dose-dependent manner as shown in FIG. 3, and IC₅₀ value thereof was 63.2±4.5 μM, indicating that it possessed the directly inhibitory function for PRMT5 methyltransferase.

### 3. Compound 3039-0164 Decreases the Symmetric Dimethylation Level of Histone 3 (H3R8me2s).

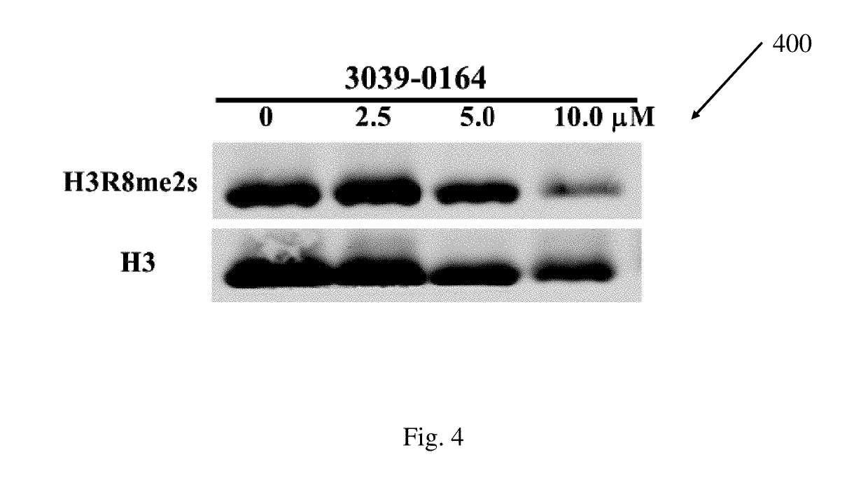

PRMT5-driven methylation of arginine residues lead to H3R8me2s. H3R8me2s expression in A549 cells was measured with or without compound 3039-0164 respectively by western blot analysis. After the treatment of compound 3039-0164 for 24 h, H3R8me2s expression was significantly decreased, as shown in FIG. 4, indirectly reflecting that compound 3039-0164 inhibits PRMT5 methyltransferase activity.

4. Compound 3039-0164 Down-Regulates Oncogene FGFR3 and eIF4E Expression.

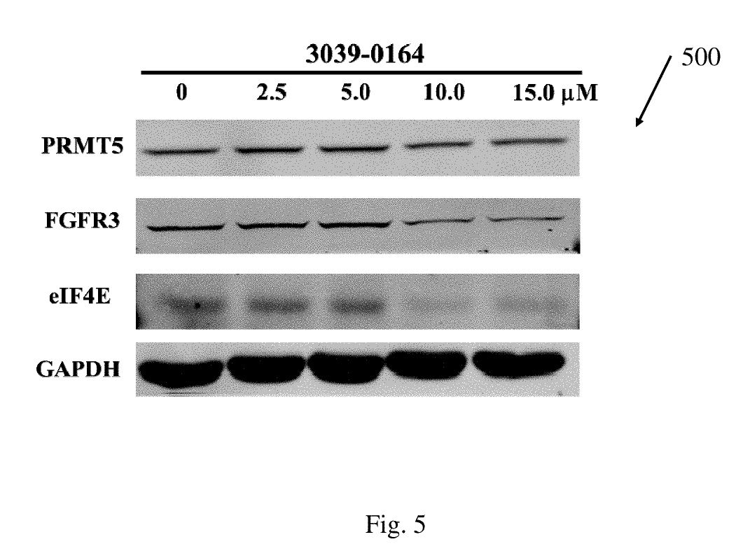

FGFR3 and eIF4E play key roles in cell proliferation and tumorigenesis. PRMT5 exerts its function by regulating the expression of target genes, including oncogene FGFR3 and eIF4E. To evaluate the effect of compound 3039-0164 on FGFR3/eIF4E, protein expression levels of two target genes in A549 cells with or without compound 3039-0164 treatment were monitored respectively. FIG. 5 showed that FGFR3 and eIF4E expression levels were significantly decreased in A549 cells after being treated with 10 μM compound 3039-0164 for 24 hours, indicating that compound 3039-0164 down-regulates FGFR3 and eIF4E expression by inhibiting PRMT5.

5. Compound 3039-0164 Suppresses the Activation of AKT, ERK and mTOR.

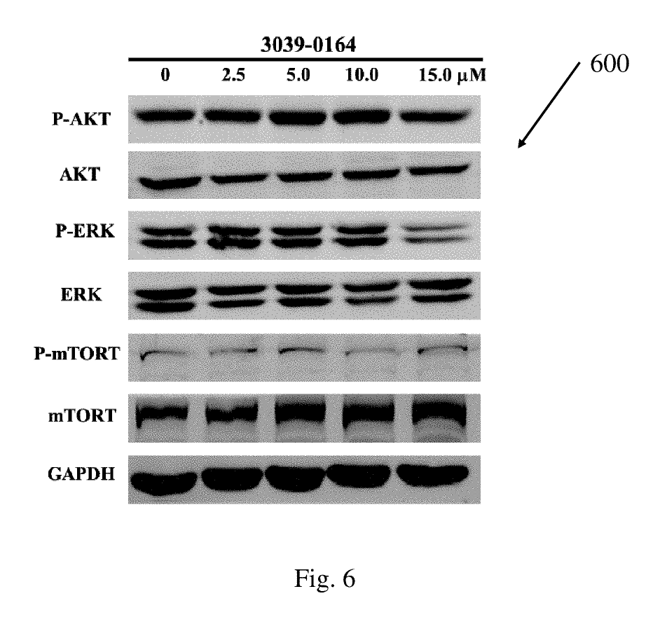

FGFR3 is one of the receptors that promote cell survival by stimulating PI3K/AKT/mTOR signaling, and can activate AKT and ERK in human cancers. The effect of compound 3039-0164 on the activation of AKT, ERK and mTOR in A549 cell was measured by western blot analysis. As shown in FIG. 6, compound 3039-0164 significantly decreased the levels of phosphorylated AKT, ERK and mTOR in A549 cell. The results implied that compound 3039-0164 suppressed the activation of PI3K/AKT/mTOR and ERK signaling in NSCLC cell.

### 6. Binding Mode of Compound 3039-0164 Inhibitor and PRMT5 Protein

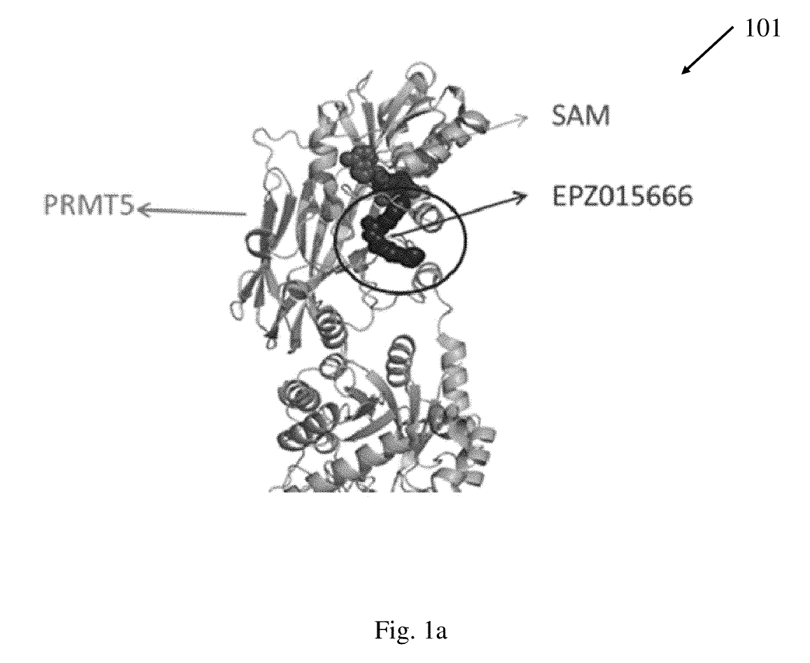

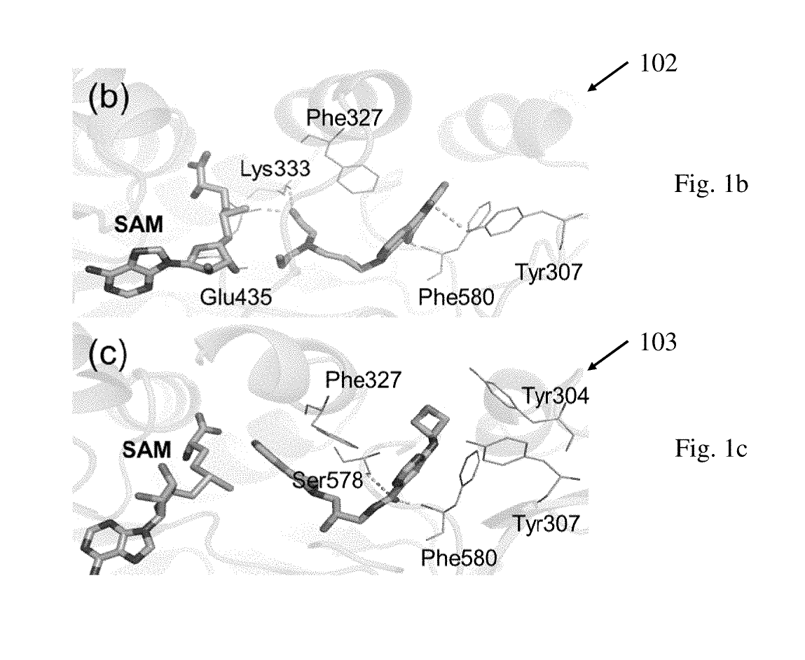

A crystal structure 101 of PRMT5 complexed with SAM and EPZ015666 (PDB ID:4×61) was used for modeling a binding mode between Formula I and PRMT5. The result from molecular docking calculation showed that compound 3039-0164 binds to PRMT5 with a low glide score of −9.26 kcal/mol. When interacting with PRMT5, compound 3039-0164 buried in a hydrophobic pocket composed of Tyr307, Phe327, Lys333, Glu435 and Phe580, as shown in the conformation 102 of FIG. 1b. Comparing the conformation 102 of compound 3039-0164 with the conformation 103 of the co-crystalized EPZ015666 (shown in FIG. 1c), it was found that compound 3039-0164 adopted similar conformation to EZP015666 in the complex. Apart from the hydrogen bond with residue Phe580 as observed in EPZ015666-PRMT5 complex, compound 3039-0164 also formed three additional hydrogen bonds with Tyr307, Lys333 and Glu435. Additional hydrogen bonds indicates a stronger affinity to PRMT5 and thus a more potent inhibitor.

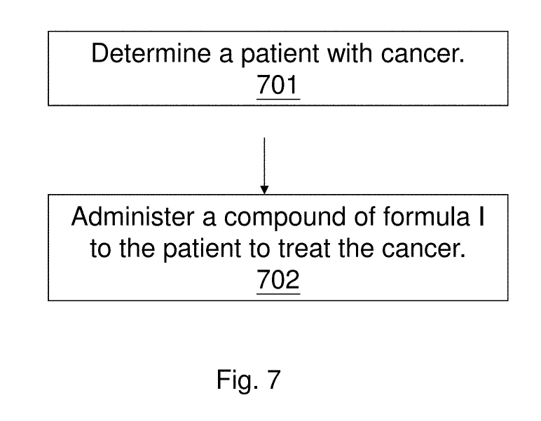

FIG. 7 is a method to treat cancer in a patient.

Block 701 states determine a patient with cancer.

In one example embodiment, the cancer is lymphomas, breast cancer, lung cancer, colorectal cancer or glioblastoma. In a further embodiment, the cancer is non-small cell lung cancer. In another example embodiment, the non-small cell lung cancer can be determined whether the patient suffers from cancer using methods such as chest X-ray, multi-detector computerized tomography scan, magnetic resonance imaging, positron emission tomography, fine and core needle biopsies of the lung, bronchoscopy, endobronchial ultrasound, navigational bronchoscopy, etc.

Block 702 states administer the compound of Formula I to the patient to treat the cancer.

In one example embodiment, the compound is administered directly or in pharmaceutical compositions along with suitable carriers or excipients. In one example embodiment, suitable routes of administration may, for example, include oral, rectal, transmucosal, nasal, or intestinal administration and parenteral delivery. The compound or the pharmaceutical composition that includes the compound can be administered locally. For example, the compound can be delivered via injection or in a targeted drug delivery system, such as a depot or sustained release formulation.

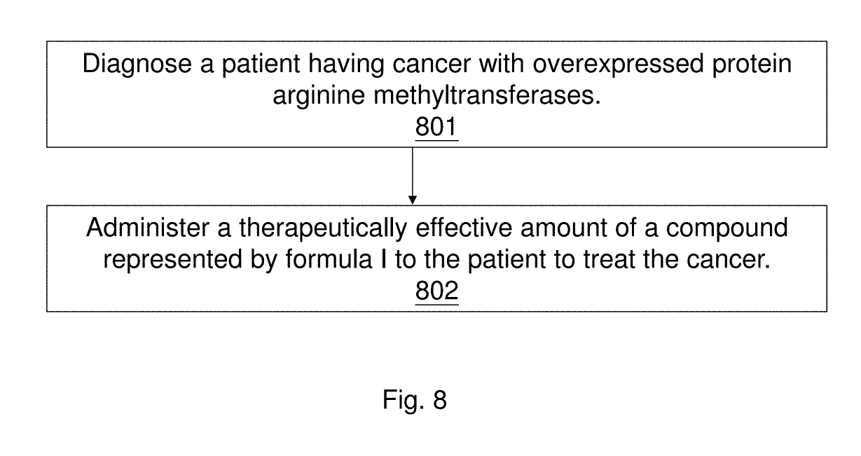

FIG. 8 is a method to treat cancer in a patient in need of such treatment in accordance with an example embodiment.

Block 801 states diagnose the patient having cancer with overexpressed PRMTs.

In an example embodiment, the protein arginine methyltransferase is PRMT5. In another embodiment, the expression level of PRMT5 is measured to determine whether a patient has cancer with overexpressed PRMTs.

Block 802 states administer a therapeutically effective amount of the compound represented by Formula I to the patient to treat the cancer with overexpressed PRMTs.

In one example embodiment, the compound is administered directly or in pharmaceutical compositions along with suitable carriers or excipients. In one example embodiment, suitable routes of administration may, for example, include oral, rectal, transmucosal, nasal, or intestinal administration and parenteral delivery. The compound or the pharmaceutical composition that includes the compound can be administered locally. For example, the compound can be delivered via injection or in a targeted drug delivery system, such as a depot or sustained release formulation.

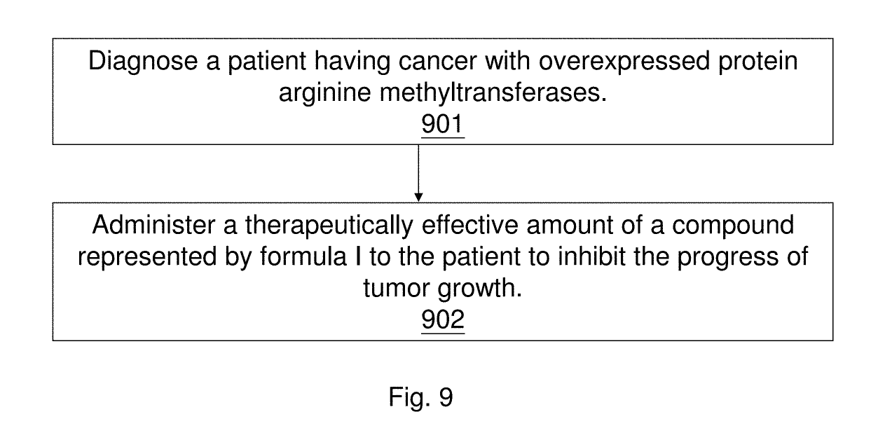

FIG. 9 is a method to inhibit progress of tumor growth in a patient with cancer.

Block 901 states diagnose a patient have cancer with overexpressed PRMTs.

In an example embodiment, the protein arginine methyltransferase is PRMT5. In another embodiment, the expression level of PRMT5 is measured to determine whether a patient has cancer with overexpressed PRMTs.

Block 902 states administer a therapeutically effective amount of the compound represented by Formula I to the patient with the cancer to inhibit progress of tumor growth.

In an example embodiment, the compound is administered directly or in the form of pharmaceutical compositions with suitable carriers or excipients. In one example embodiment, suitable routes of administration may, for example, include oral, rectal, transmucosal, nasal, or intestinal administration and parenteral delivery. The compound or the pharmaceutical composition that includes the compound can be administered locally. For example, the compound can be delivered via injection or in a targeted drug delivery system, such as a depot or sustained release formulation.

As used herein, the term “patient” encompasses mammals and non-mammals. Examples of mammals include, but are not limited to, humans, chimpanzees, apes monkeys, cattle, horses, sheep, goats, swine; rabbits, dogs, cats, rats, mice, guinea pigs, and the like. Examples of non-mammals include, but are not limited to, birds, fish and the like.

As used herein, the term “therapeutically effective amount” refers to any amount of a compound which, as compared to a corresponding patient who has not received such amount, results in improved treatment, healing, prevention, or amelioration of a disease, disorder, or side effect, or a decrease in the rate of advancement of a disease or disorder.

As used herein, the term “PRMT5 inhibitor” refers to a molecule that binds to PRMT5 and decreases the activity thereof.

As used herein, the term “normal cells” refers to the cells which do not exhibit uncontrolled cell growth or the ability to metastasize. The term “normal cells” also include but not limited to “benign cells”, “non-cancer cells” and “non-malignant cells”.

As used herein, the term “overexpress” or “overexpression” refers to too many copied of a protein is made.

The term “pharmaceutically acceptable excipient” refers to pharmacologically inactive substances that are added to a pharmaceutical preparation in addition to the active pharmaceutical ingredient. Pharmaceutically acceptable excipients may take the function of vehicle, diluent, release, disintegration or dissolution modifying agent, absorption enhancer, stabilizer or a manufacturing aid among others.

As used herein, the term “treat,” “treating” or “treatment” refers to methods of alleviating, abating or ameliorating a disease or condition symptoms, preventing additional symptoms, ameliorating or preventing the underlying metabolic causes of symptoms, inhibiting the disease or condition, arresting the development of the disease or condition, relieving the disease or condition, causing regression of the disease or condition, relieving a condition caused by the disease or condition, or stopping the symptoms of the disease or condition either prophylactically and/or therapeutically.

As used herein, the term “administration” or “administering” of the patient compound refers to providing a compound of an example embodiment and/or prodrugs thereof to a patient in need of treatment.

As used herein and in the claims, “comprising” means including the following elements but not excluding others.

1.-13. (canceled)

14. A method of treating non-small cell lung cancer in a patient in need thereof, comprising:
administering a therapeutically effective amount of a compound to the patient to treat the non-small cell lung cancer,
wherein the compound is represented by Formula I:

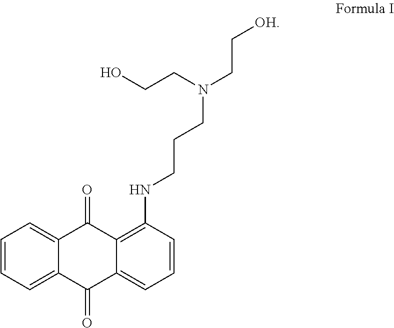 [SMILES: O=C1c2ccccc2C(=O)c2c(NCCCN(CCO)CCO)cccc21]

15. The method of claim 14, wherein the non-small cell lung cancer has overexpressed protein arginine methyltransferase 5 (PRMT5).

16. The method of claim 15, wherein the compound inhibits PRMT5 such that the non-small cell lung cancer is treated.

17. A method of treating non-small cell lung cancer in a patient in need thereof, comprising:
detecting whether the patient with the non-small cell lung cancer has overexpressed protein arginine methyltransferase 5 (PRMT5);
diagnosing the patient whose non-small cell lung cancer cells have overexpressed PRMT5; and
administering a therapeutically effective amount of a compound to the patient to treat the non-small cell lung cancer with overexpressed PRMT5,
wherein the compound is represented by Formula I:

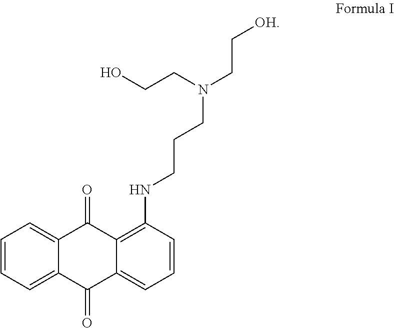 [SMILES: O=C1c2ccccc2C(=O)c2c(NCCCN(CCO)CCO)cccc21]

18. The method of claim 17, wherein the compound treats the non-small cell lung cancer by inhibiting PRMT5.
